(19) United States
(12) Patent Application Publication
(10) Pub. No.: US 2007/0020753 A1
(43) Pub. Date: Jan. 25, 2007

(54) Condensation reducing/eliminating container

(75) Inventor: John J. Finer, Wooster, OH (US)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: The Ohio State University, Columbus, OH (US)

(21) Appl. No.: 11/489,279

(22) Filed: Jul. 19, 2006

Related U.S. Application Data
(60) Provisional application No. 60/700,454, filed on Jul. 19, 2005.

(51) Int. Cl. C12M 1/22

(52) U.S. Cl. 435/305.1; 435/305.4

(57) ABSTRACT

The present invention provides methods for reducing the amount of condensation in a container by providing a container comprising a lid, wherein the amount of condensation in the container is reduced by: increasing all or a portion of the thickness of the lid; insulating the lid; or combinations thereof. The present invention also provides containers comprising lids that provide a reduction in the amount of condensation resulting from temperature fluctuation.

### CROSS REFERENCE TO RELATED APPLICATION

This patent application claims the benefit of U.S. Provisional Application Ser. No. 60/700,454, filed Jul. 19, 2005.

### FIELD OF THE INVENTION

The present invention is directed to methods for reducing or eliminating the amount of condensation in a container. The methods comprise providing a container comprising a lid, wherein the amount of condensation in the container is reduced by: increasing the thickness of the lid; insulating the lid; or combinations thereof. The present invention is also directed to containers comprising a lid that provide a reduction in the amount of condensation resulting from temperature fluctuation, wherein the amount of condensation in the container is reduced by: increasing the thickness of the lid; shortening the walls of the lid; insulating the lid; or combinations thereof.

### BACKGROUND

Condensation on the inside lid of containers, including Petri dishes and culture vessels, is a universal problem in the laboratory. Condensation problems result when the temperature of the inner surface of the lid of a container falls below the temperature inside the container itself. Condensation can take the form of either a light fog or large liquid droplets. When large liquid droplets form, they can drop from the lid onto the contents of the container, displacing the contents and creating liquid soaked microenvironments. When a light condensation is formed, the contents of the container are often obscured, which makes critical observations difficult and photodocumentation of the contents of the container very frustrating to achieve.

Condensation forms on the lids and sides of containers whenever a temperature differential occurs; such as, for example, if the temperature of the container lid is below the temperature of the contents of the container. This occurs most often when the temperature in a room fluctuates and falls, even by as little as 0.1° C. If a room cools, the container will retain some heat while the air temperature within the room and surrounding the container is lowered. Moisture or condensation will form on the relatively cool inner lid surface in the high humidity conditions that exist within the container.

The temperature of the inside of a container can also be increased and condensation results if the contents absorb sufficient light to generate heat energy. Placement of containers directly on dark surfaces in the light can give similar results, as the dark surface absorbs light energy, warming the container. In addition, bottom heat, generated from lights or equipment under shelves holding containers, can also lead to a temperature differential and condensation.

Even though many laboratories have very good temperature control, small fluctuations in temperature occur and, every time this happens, additional condensation forms. The end results can be layers of condensation, eventually forming water droplets on the lids of the containers. Some laboratories blow heated air over the tops of the containers or force cool air underneath the containers, which effectively keeps the lid temperature above the container temperature, eliminating condensation problems. However, most laboratories do not have these types of facilities.

### SUMMARY OF THE INVENTION

The present invention provides methods for reducing the amount of condensation in a container comprising providing a container including a lid. The amount of condensation in the container is reduced by: increasing the thickness of the lid; insulating the lid, for example with a piece of insulating material; or combinations thereof.

The present invention also provides containers including a lid that provides a reduction in the amount of condensation resulting from temperature fluctuation. The amount of condensation in the container is reduced by: increasing the thickness of the lid; shortening the walls of the lid; insulating the lid with a piece of insulating material or combinations thereof. In one embodiment, the container is a culture container. In another embodiment, the container is a Petri dish. In yet another embodiment, the container is a culture dish. In a further embodiment, the container is a vented dish.

### DETAILED DESCRIPTION OF THE FIGURES

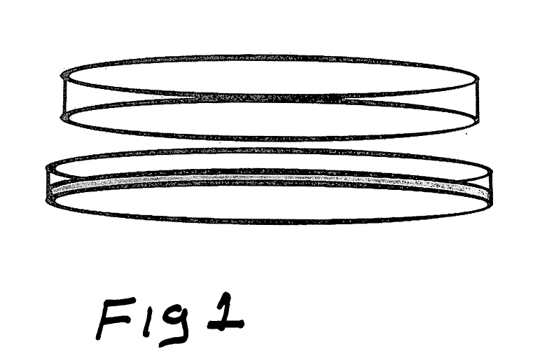

FIG. 1. A Petri dish with a thickened lid design.

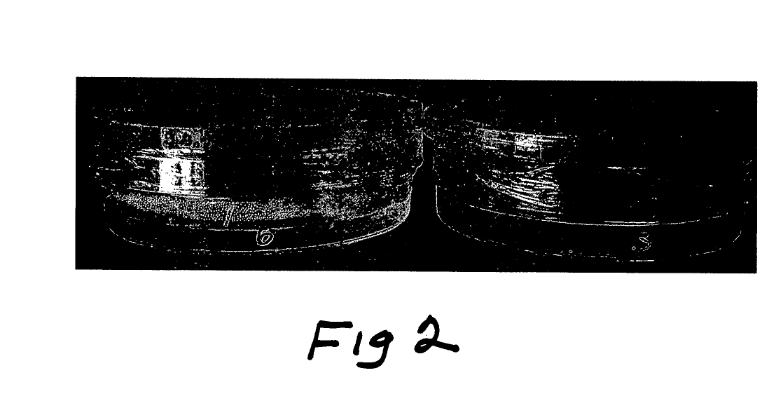

FIG. 2. A polymer disc is placed on top of a standard Petri dish lid (left) or in place of standard Petri dish lid (right); and

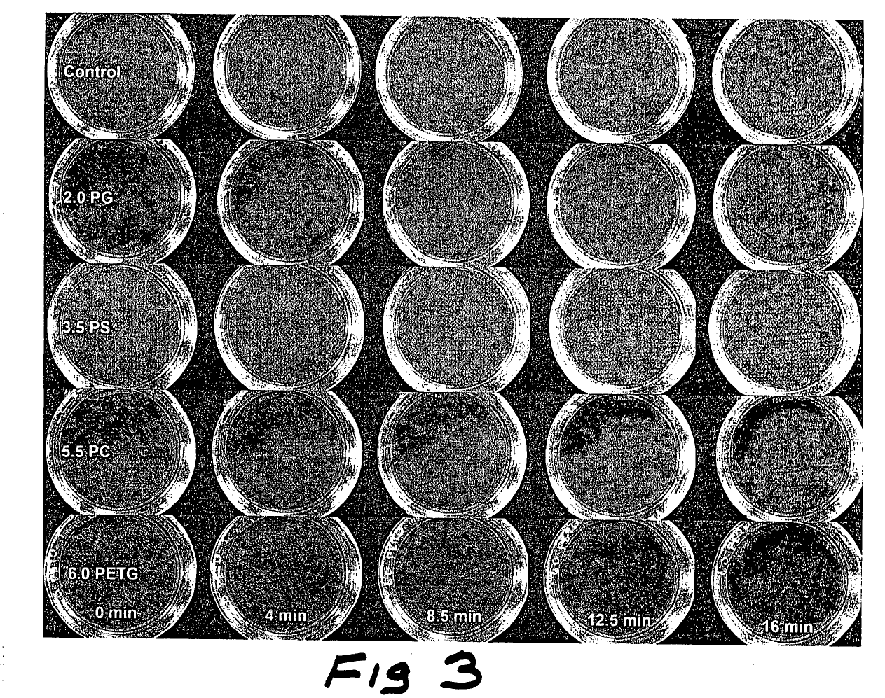

FIG. 3. A composite image of Double Petri dish lids, consisting of a control and 4 different polymers, of 4 different thicknesses, taken over a 16 minute period. Condensation in this short-term experiment was subtle but is clearly observable. PG=Plexiglas, PS=polystyrene, PC=polycarbonate, PETC=an additional form of polycarbonate.

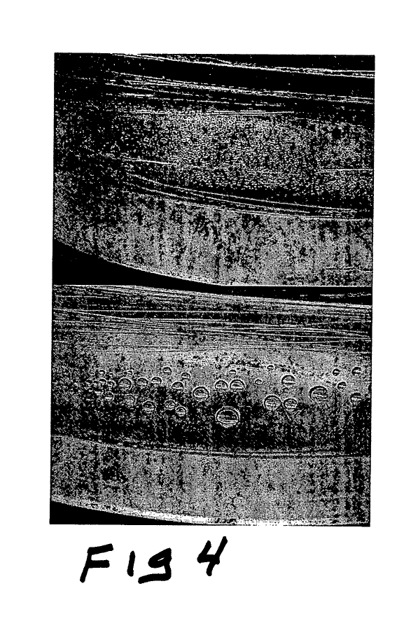

FIG. 4. Petri dishes displaying condensation on the dishes' side.

### DETAILED DESCRIPTION OF THE INVENTION

The inventor has discovered a way to reduce or eliminate condensation from forming on the surface of containers, such as Petri dishes and culture containers. By replacing the standard lid with other types of transparent materials in varying thicknesses, temperature differentials can be buffered; offering protection from fluctuations in temperature such that condensation does not form on the lid surface. The thicker materials insulate the lid from temperature fluctuations. If the temperature of the inside lid of the container is not reduced, condensation will not form

Various materials possess different thermal conductivities. Materials with high thermal conductivities allow heat to transfer quickly while materials with low thermal conductivities act as heat insulators. Most metals and glass have high thermal conductivities. Many plastics have low thermal conductivities and are good insulators. Petri dishes, for example, were originally manufactured from glass but are now manufactured primarily from polystyrene, which has a relatively low thermal conductivity. If the lids of Petri dishes (top part only), or all or a portion of the surface of other culture containers were manufactured with increased thickness or modified to include a pocket of insulating air, condensation problems could be reduced or eliminated. In addition, placement of a small piece of insulating material inside or on top of a Petri dish lid (outside of the container) yields similar results.

The invention does not require large changes in manufacturing basics. Containers can still be manufactured from polystyrene, which is optically clear, inexpensive and a good insulator (low thermal conductivity). The containers can still be made using molds and the bottom of the containers, for example the bottom of Petri dishes, can be produced exactly the same as they are now. The main difference is the top portion of the lid of a Petri dish or the top of another culture container. The new lid will be thicker on all or a portion of the top surface than they are in the prior art. The sides of the lid can remain the same thickness or the sidewalls of the lid may be shortened. While not wishing to be bound by theory, it is believed that if the sidewalls are shortened, the sidewalls will cool more quickly and, if any condensation does form, it will occur there. Condensation occurs at the thinnest point of the container, where insulation is minimal and the lid walls do not provide a second layer of insulating polymer material (See FIG. 4, left—condensation is below the lid sidewall, on the bottom sidewall). Condensation on the side of the container is preferred as it will not obscure the container's contents from view and drops of condensation will be reabsorbed into the medium if they fall down the container side. However, as long as all or a portion of the top of the container is thicker, any water droplets, if formed, will slide down the sidewalls and be reabsorbed by the medium.

Alternatively to basic changes in Petri dish or culture container design, insulating materials can be placed either on top of the lid of the container or on the inside top of the lid of the container. Placement of insulating materials on the inside top of the lid of the container has the advantage that an additional pocket of air can be sandwiched in between the two surfaces, providing more insulation from temperature fluctuations.

### EXAMPLES

The inventor initially evaluated discs of different clear plastic polymers and glass. There are a few different sizes of Petri dishes. The most common Petri dish size is 100×15 mm. For most of the experiments, the inventor used a 100×25 mm size. For the smaller dishes, it may be even more important to reduce the side walls of the lid, as the dish is so short.

The discs were cut to the same size as the Petri dish lid and were either sterilized and used in place of lids or placed on top of manufactured lids. Under conditions of temperature differential, moisture or condensation forms on the relatively cool inner lid surface.

To quantify condensation of the different lids, Petri dishes with modified lids were initially maintained in a laboratory where the temperature was maintained at 25° C. and transferred to conditions where the temperature could be gradually lowered. At specific time points, images of the Petri dishes were collected using a digital camera, mounted on a copy stand. Images were collected one dish at a time and as quickly as possible. The time of image collection was recorded. Condensation was quantified using Adobe Photoshop® by selecting a 1000×1000 pixel region of the image and reading the Mean value of intensity in the Histogram palette. The initial time point value was subtracted from the timed values, to yield a value for increase in condensation over time.

The four different polymers tested were Plexiglas, Polycarbonate, Polystyrene, and PETG, which is a form of Polycarbonate. Glass (borosilicate) was also initially evaluated, but its insulating properties were very poor and condensation comparisons of the glass to the polymers were difficult due to the different forms of condensation. The thermal conductivity of the four different polymers is slightly different. Those values are shown in Table 1.

TABLE 1
Thermal Conductivity Values

| Polymer | Thermal Conductivity* |
|---|---|
| PG - Plexiglas | 1.32-1.67 |
| PC - Polycarbonate | 1.32-1.46 |
| PS - Polystyrene | 0.83-1.34 |
| PETG - form of PC | 1.32 |
| Glass (borosilicate) | 7.63 |

*for thermal conductivity values, lower values indicate low heat transfer and good insulation capacity. Thermal conductivity values were obtained from MatWeb.com
Units: BTUs lost - in/hr/ft² with a 1° F. temp differential

Polystyrene has the lowest thermal conductivity, indicating that it is the best insulator. Polystyrene is also the standard material used in the manufacture of Petri dishes.

While conducting the short term experiments, the inventor encountered some problems. These problems included contamination of the plates, scratching the lids, oil from hands on the lids, uneven cooling, the camera focusing on its reflection, and dust on the dish lids.

The polymer lids were initially used in place of the standard manufactured lids. They needed to be cleaned for visual clarity and then sterilized (with ethanol) as the Petri dishes contained a standard culture medium, which would support the growth of bacterial and fungal contaminants if left untreated. Cleaning and sterilizing the dish lids using 70% or 95% ethanol was not successful. Latex gloves were used when handling alcohol-saturated wipes but microabrasion occurred from wiping the polymers with Kimwipes or paper towels. This scratching interfered with the image analysis and appeared to for a nucleus for condensation. The solution to this problem was to use the original dish lid as an inner lid then place the lid being tested on top of the original dish lid (FIG. 2). This eliminated streaking and scratching. The original Petri dish lid inner surface provided a consistent surface for studies on condensation.

Experiments were set up by placing a set of Petri dishes with the modified lids in the laboratory, next to a window (in cool weather) and allowing the Petri dishes to slowly cool for 20 minutes. Temperature changes were monitored with a digital thermometer and temperature changes over the 20 minute experiments were 1.6-3.5° C. This temperature change is higher than would normally be encountered in a laboratory environment but the condensation response was more easily documented under these conditions.

A digital camera was used in a full manual mode to prevent the camera from focusing on its reflection instead of the Petri dish lid. In addition, a hood, consisting of a black sheet of fabric placed in front of the camera (with a hole cut for the camera lens), effectively eliminated background lighting and the remaining reflection problems. A piece of black velvet fabric was placed under the dishes for a high contrast background and the camera was mounted on a copy stand, which used 4×50 W bulbs for consistent illumination.

Dust, which could interfere with image analysis, was eliminated from the surface of the dish lids by wiping off the lids with a black felt glove between each image collection.

The values presented in Table 2 are taken from images that were analyzed separately but are presented collectively (as a smaller sample) in FIG. 3. Table 2 values represent the changes in gray value intensity measurements from time 0. The experiment was repeated 5 times. Although the condensation shown in the composite image (FIG. 3) appears at first to be subtle, it is easily quantifiable from the original image and gray value determinations are clear. Condensation of moisture on the lids of all of the Petri dishes increased over time as the temperature surrounding the Petri dishes decreased. The control dish lid, without the additional polymer disc, displayed the highest level of condensation while the thicker dish lids displayed the lowest increase. The polycarbonate lids, did not provide as much protection from condensation as the other polymers. Polystyrene, the component polymer of Petri dishes, appears to provide adequate protection from condensation, with the thickened lid.

TABLE 2
Double Lid - Condensation (mean gray value intensity)
using different Petri dish lids

| Lid Thickness (mm) | Range of Times for Image Collection - minutes | | | |
|---|---|---|---|---|
| and Type of polymer | 4-6:30 | 7-13:00 | 12-16:30 | 15:45-20 |
| Control | 23.32 | 35.19 | 41.71 | 47.46 |
| 1.0 Plexiglas | 12.68 | 23.20 | 29.35 | 35.78 |
| 2.0 Plexiglas | 6.97 | 13.21 | 17.04 | 24.11 |
| 2.0 PETG | 13.61 | 21.79 | 28.59 | 34.50 |
| 2.5 Plexiglas | 4.57 | 12.99 | 19.11 | 23.13 |
| 3.5 Polystyrene | 5.08 | 9.94 | 13.61 | 15.95 |
| 4.0 Polycarbonate | 9.28 | 15.05 | 20.99 | 26.34 |
| 4.5 Plexiglas | 0.98 | 2.75 | 7.87 | 10.60 |
| 5.5 Plexiglas | 0.58 | 2.38 | 7.49 | 11.48 |
| 5.5 Polycarbonate | 1.72 | 2.94 | 8.64 | 12.67 |
| 6.0 PETG | 0.62 | 1.78 | 2.66 | 4.71 |

What is claimed is:

1. A method of reducing the amount of condensation in a container comprising providing a container comprising a lid, wherein the amount of condensation in the container is reduced by increasing the thickness of the lid.

2. A method of reducing the amount of condensation in a container comprising providing a container comprising a lid, wherein the amount of condensation in the container is reduced by insulating the lid.

3. The method of claim 2, wherein the insulating material is located inside the lid, on the top of the lid or a combination thereof.

4. The method of claim 2, wherein the lid further comprises a pocket of insulating air.

5. The method of claim 1, wherein the container is a Petri dish, a culture dish, a culture container or a vented dish.

6. The method of claim 1, wherein all or a portion of the thickness of the lid is increased.

7. A container comprising a lid that provides a reduction in the amount of condensation resulting from temperature fluctuation, wherein the amount of condensation in the container is reduced by increasing the thickness of the lid.

8. The container of claim 7, wherein the side walls of the lid are shortened.

9. The container of claim 7, wherein the container is a Petri dish, a culture dish, a culture container or a vented dish.

10. The container of claim 7, wherein all or a portion of the thickness of the lid is increased.

11. A container comprising a lid that provides a reduction in the amount of condensation resulting from temperature fluctuation, wherein the amount of condensation in the container is reduced by insulating the lid.

12. The method of claim 11, wherein the insulating material is located inside the lid, on top of the lid or a combination thereof.

13. The container of claim 11, wherein the lid further comprises a pocket of insulating air.
